(19) United States
(12) Patent Application Publication
(10) Pub. No.: US 2026/0325594 A1
(43) Pub. Date: Oct. 22, 2026

(54) PREPARATION METHOD FOR GINKGO BILOBA EXTRACT AND PRODUCT AND APPLICATION THEREOF

(71) Applicant: Beijing Rongsen Jiuding Technology Co., Ltd., Beijing (CN)

(72) Inventors: Yuan Liu, Beijing (CN); Yixia Li, Beijing (CN); Jinrong Liu, Beijing (CN); Jingdong Li, Beijing (CN)

(21) Appl. No.: 19/468,762

(22) Filed: Feb. 3, 2026

(30) Foreign Application Priority Data
Feb. 6, 2025 (CN) 202510132573.8

(51) Int. Cl. A61K 36/16; A61K 8/9771; A61Q 17/00; A61Q 19/00

(52) U.S. Cl. CPC A61K 36/16; A61K 8/9771; A61Q 17/00; A61Q 19/007; A61K 2236/19; A61K 2236/331; A61K 2236/333; A61K 2236/39; A61K 2236/51; A61K 2236/53; A61K 2800/522; A61K 2800/805; C12Y 302/01008; C12Y 302/01015; C12Y 302/01021

(57) ABSTRACT

A preparation method for Ginkgo biloba extract, a product and an application thereof, comprising mixing Ginkgo biloba raw material with a composite enzyme for enzymatic hydrolysis; followed by alcohol extraction and filtration, first separation and purification, fine extraction, second separation and purification, and flocculation and drying to obtain the Ginkgo biloba extract; wherein the composite enzyme comprises a combination of β-glucosidase, xylanase, hemicellulase, and pectinase. The invention employs a specific extraction and separation process, and further utilizes a specific enzyme combination. It has been found that the synergistic interaction of these four enzymes can enhance the enzymatic extraction efficiency, thereby improving the moisturizing, anti-inflammatory, and antioxidant effects of the extract; the resulting extract exhibits excellent water solubility, making it highly suitable for the preparation of moisturizing and antioxidant products; additionally, it is mild and non-irritating, with good stability.

### TECHNICAL FIELD

The invention belongs to the technical field of lant extracts, in particular to a preparation method for Ginkgo biloba extract, a product and an application thereof.

### BACKGROUND ART

Ginkgo biloba is the dried leaf of Ginkgo biloba L., a plant in the family Ginkgoaceae. They are harvested in autumn while the leaves are still green and dried promptly; the plants are generally cultivated. The Ginkgo tree is a large deciduous tree characterized by long branches and slow-growing, spur-like short branches. The leaves are alternate, scattered radially on long branches, and clustered in groups of 3 to 5 on short branches. They feature long, slender petioles and a fan-shaped lamina, light green on both sides. The broad apex is more or less notched or 2-lobed, measuring 5 to 8 (up to 15) cm in width, with numerous dichotomously branched parallel fine veins.

Medicinal Value and Efficacy: Ginkgo biloba possesses high medicinal value, offering the following therapeutic effects: promoting blood circulation and removing blood stasis; dredging collaterals and relieving pain; astringing the lungs and relieving asthma; resolving turbidity and lowering blood lipids. It can serve as pharmaceutical components for the relief or treatment of conditions such as blood stasis obstructing collaterals, chest obstruction and cardiac pain, stroke-induced hemiplegia, lung-deficiency cough and asthma, and hyperlipidemia. Ginkgo biloba is rich in various active substances, including flavonoids and terpene lactones. These compounds exhibit significant pharmacological actions, such as: dilating blood vessels and protecting vascular endothelial tissues; regulating blood lipids and protecting low-density lipoprotein (LDL); inhibiting PAF (Platelet-Activating Factor) and suppressing thrombus formation and scavenging free radicals.

Furthermore, Ginkgo biloba extract holds significant application value within the cosmetics industry. Peroxides and free radicals present on the skin surface are primary drivers that accelerate skin browning, promote aging, and lead to the formation of age spots. To delay oxidative skin aging, these substances must be cleared promptly. Ginkgo biloba extract exhibits Superoxide Dismutase (SOD) activity, making it an excellent free radical scavenger with the added benefit of promoting blood circulation. Consequently, incorporating this extract into skincare products can moisturize the skin, enhance radiance, and reduce melanin formation. Therefore, it is essential to develop a novel, safe, environmentally friendly, and high-purity preparation method for Ginkgo biloba extract.

### SUMMARY OF THE INVENTION

To address the shortcomings of existing technologies, the invention aims to provide a preparation method for Ginkgo biloba extract, a product and an application thereof

To achieve this objective, the invention adopts the following technical solution:

in a first aspect, the invention provides a preparation method for Ginkgo biloba extract, comprising the following steps:
S1, rough extraction step: mixing Ginkgo biloba raw material with a composite enzyme for enzymatic hydrolysis; after the enzymatic hydrolysis is completed, using an ethanol aqueous solution for reflux extraction; concentrating the reflux liquid to remove ethanol; then mixing the concentrated solution with water; heating it; letting it stand; filtering the mixture; and collecting filtrate to obtain a rough extract;
S2, first separation and purification step: enriching the rough extract using macroporous resin to obtain a first eluent;
S3, fine extraction step: concentrating and drying the first eluent to obtain Ginkgo biloba rough extract, then using an ethanol aqueous solution for hot immersion extraction, cooling, filtering, and collecting filtrate to obtain a fine extract;
S4, second separation and purification step: enriching the fine extract sequentially using polyamide resin and macroporous resin to obtain a second eluent;
S5, flocculation step: standing and filtering the second eluent, then collecting filtrate, mixing the filtrate with a flocculant to obtain a flocculation treatment liquid;
S6, drying step: centrifuging and filtering the flocculation treatment liquid, collecting and drying filtrate to obtain Ginkgo biloba extract;
wherein the composite enzyme comprises a combination of β-glucosidase, xylanase, hemicellulase, and pectinase.

The invention creatively found the composite enzyme composed of four enzymes: β-glucosidase, xylanase, hemicellulase, and pectinase, which can interact and positively influence each other to enhance the extraction efficiency of Ginkgo biloba raw materials, thereby increasing the active ingredients in the extract products.

Moreover, the preparation method for the Ginkgo biloba extract involved in the invention innovatively employs a specific extraction and separation purification process on the Ginkgo biloba treatment solution after enzymatic pretreatment; combined with subsequent flocculant treatment, this method can efficiently extract the effective components from the Ginkgo biloba, resulting in a high-purity and high-activity Ginkgo biloba extract product that possesses excellent water solubility and can be further applied in cosmetics to enhance moisturizing, anti-inflammatory, and antioxidant effects.

Preferably, a mass ratio of β-glucosidase, xylanase, hemicellulase, and pectinase is (1-5):(2-4):(2-6):(0.5-3).
Wherein, specific values in “1-5” may include 1, 1.5, 2, 2.5, 3, 3.5, 4, 4.5, 5, etc.;
specific values in “2-4” may include 2, 2.2, 2.5, 2.8, 3, 3.2, 3.5, 3.8, 4, etc.;
specific values in “2-6” may include 2, 2.5, 3, 3.5, 4, 4.5, 5, 5.5, 6, etc.;
specific values in “0.5-3” may include 0.5, 0.8, 1, 1.2, 1.5, 1.8, 2, 2.2, 2.5, 2.8, 3, etc. Other specific points within this value range can also be selected, which will not be reiterated here.

In the invention, when dosage ratios of each enzyme in the composite enzyme combination used are within the aforementioned specific ranges, the enzymatic extraction effect on Ginkgo biloba is more favorable.

Preferably, the Ginkgo biloba raw material is mixed with water at a solid-to-liquid ratio of 1:(8-15) g/mL; for example, it can be 1:8 g/mL, 1:9 g/mL, 1:10 g/mL, 1:11 g/mL, 1:12 g/mL, 1:13 g/mL, 1:14 g/mL, 1:15 g/mL, etc. Other specific point values within this range can also be selected, which will not be reiterated here.

In the invention, when the ratio of Ginkgo biloba raw material to water is within the aforementioned specific range, it exhibits a more favorable enzymatic effect.

Preferably, an amount of the composite enzyme added is 0.1‰-0.5‰ of a mass of the Ginkgo biloba material; for example, it can be 0.1‰, 0.15‰, 0.2‰, 0.25‰, 0.3‰, 0.35‰, 0.4‰, 0.45‰, 0.5‰, etc. Other specific point values within this range can also be selected, which will not be reiterated here.

In the invention, when the amount of the composite enzyme added is within the aforementioned specific range, it exhibits a more favorable extraction effect on the Ginkgo biloba raw material.

Preferably, a temperature for enzymatic hydrolysis is 40-60° C., for example, it can be 40° C., 41° C., 42° C., 43° C., 44° C., 45° C., 46° C., 47° C., 48° C., 49° C., 50° C., 52° C., 55° C., 58° C., 59° C., 60° C., etc.; a duration is 0.5-2 hours, for example, it can be 0.5 h, 0.8 h, 1 h, 1.2 h, 1.5 h, 1.8 h, 2 h, etc. Other specific point values within this range can also be selected, which will not be reiterated here.

Preferably, in S1, a ratio of the Ginkgo biloba raw material to the ethanol aqueous solution is 1:(4-10) g/mL; for example, it can be 1:4 g/mL, 1:5 g/mL, 1:6 g/mL, 1:7 g/mL, 1:8 g/mL, 1:9 g/mL, 1:10 g/mL, etc. Other specific point values within this range can also be selected, which will not be reiterated here.

Preferably, in S1, a volume percentage of ethanol in the ethanol aqueous solution is 30%-90%; for example, it can be 30%, 40%, 50%, 60%, 70%, 80%, 90%, etc. Other specific point values within this range can also be selected, which will not be reiterated here.

Preferably, in S1, the number of reflux extraction cycles is 2-5 times, with each reflux extraction lasting 1-3 hours; for example, it can be 1 h/time, 1.2 h/time, 1.5 h/time, 2 h/time, 2.5 h/time, 3 h/time, etc. Other specific point values within this range can also be selected, which will not be reiterated here.

Preferably, in S1, a volume ratio of the concentrated solution to water is 1:(2-20); for example, it can be 1:2, 1:4, 1:6, 1:8, 1:10, 1:12, 1:14, 1:16, 1:18, 1:20, etc. Other specific point values within this range can also be selected, which will not be reiterated here.

Preferably, in S1, a temperature for mixing with water and heating is 50-100° C., for example, it can be 50° C., 55° C., 60° C., 65° C., 70° C., 75° C., 80° C., 85° C., 90° C., 95° C., 98° C., 100° C., etc.; a duration is 20-60 minutes, for example, it can be 20 minutes, 25 minutes, 30 minutes, 35 minutes, 40 minutes, 45 minutes, 50 minutes, 55 minutes, 60 minutes, etc. Other specific point values within this range can also be selected, which will not be reiterated here.

Preferably, in S1, a temperature for standing is 2-6° C., for example, it can be 2° C., 2.5° C., 3° C., 3.5° C., 4° C., 4.5° C., 5° C., 5.5° C., 6° C., etc.; a duration is 18-48 hours, for example, it can be 18 h, 19 h, 20 h, 21 h, 22 h, 24 h, 25 h, 26 h, 28 h, 30 h, 32 h, 35 h, 38 h, 40 h, 45 h, 48 h, etc. Other specific point values within this range can also be selected, which will not be reiterated here.

Preferably, in S2, a type of the macroporous resin comprises any one of AB-8 type, D101 type, or HPD100 type.

Preferably, in S2, the enrichment specifically comprises: loading the rough extract onto the macroporous resin for adsorption, eluting with water, analyzing with a 40%-90% ethanol aqueous solution, and collecting the eluent.

Preferably, an elution flow rate is 120-180 L/h, for example, it can be 120 L/h, 130 L/h, 140 L/h, 150 L/h, 160 L/h, 170 L/h, 180 L/h, etc. Other specific point values within this range can also be selected, which will not be reiterated here.

Preferably, in S3, a temperature for concentration and drying is 60-80° C.; for example, it can be 60° C., 65° C., 68° C., 70° C., 72° C., 75° C., 78° C., 80° C., etc. Other specific point values within this range can also be selected, which will not be reiterated here.

Preferably, in S3, a ratio of the Ginkgo biloba rough extract to the ethanol aqueous solution is 1:(4-10) g/mL; for example, it can be 1:4 g/mL, 1:5 g/mL, 1:6 g/mL, 1:7 g/mL, 1:8 g/mL, 1:9 g/mL, 1:10 g/mL, etc. Other specific point values within this range can also be selected, which will not be reiterated here.

Preferably, in S3, a volume percentage of ethanol in the ethanol aqueous solution is 40%-90%; for example, it can be 40%, 45%, 50%, 55%, 60%, 65%, 70%, 75%, 80%, 85%, 90%, etc. Other specific point values within this range can also be selected, which will not be reiterated here.

Preferably, in S3, a temperature for the hot immersion extraction is 40-100° C., for example, it can be 40° C., 50° C., 55° C., 60° C., 65° C., 70° C., 75° C., 80° C., 85° C., 90° C., 95° C., 98° C., 100° C., etc.; a duration is 20-60 minutes, for example, it can be 20 min, 25 min, 30 min, 35 min, 40 min, 45 min, 50 min, 55 min, 60 min, etc. Other specific point values within this range can also be selected, which will not be reiterated here.

Preferably, in S3, a cooling temperature is 2-6° C., for example, it can be 2° C., 2.5° C., 3° C., 3.5° C., 4° C., 4.5° C., 5° C., 5.5° C., 6° C., etc.; a duration is 18-48 hours, for example, it can be 18 h, 19 h, 20 h, 21 h, 22 h, 24 h, 25 h, 26 h, 28 h, 30 h, 32 h, 35 h, 38 h, 40 h, 45 h, 48 h, etc. Other specific point values within this range can also be selected, which will not be reiterated here.

Preferably, in S4, the type of macroporous resin comprises DM130.

Preferably, the enrichment specifically comprises: sequentially loading the fine extract onto the polyamide resin and macroporous resin for adsorption, eluting with water to remove impurities, analyzing with a 40%-90% ethanol aqueous solution, and collecting the eluent.

Preferably, before loading, the fine extract undergoes concentration to remove ethanol, mixing the concentrated solution with water, cooling, and filtering to collect the filtrate.

Preferably, a volume ratio of the concentrated solution to water is 1:(10-40); for example, it can be 1:10, 1:12, 1:15, 1:18, 1:20, 1:25, 1:30, 1:35, 1:40, etc. Other specific point values within this range can also be selected, which will not be reiterated here.

Preferably, a cooling temperature is 2-6° C., for example, it can be 2° C., 2.5° C., 3° C., 3.5° C., 4° C., 4.5° C., 5° C., 5.5° C., 6° C., etc.; a duration is 18-48 hours; for example, it can be 18 h, 19 h, 20 h, 21 h, 22 h, 24 h, 25 h, 26 h, 28 h, 30 h, 32 h, 35 h, 38 h, 40 h, 45 h, 48 h, etc.

Preferably, an elution flow rate for the polyamide resin is 80-150 L/h; for example, it can be 80 L/h, 85 L/h, 90 L/h, 95 L/h, 100 L/h, 110 L/h, 120 L/h, 125 L/h, 130 L/h, 135 L/h, 140 L/h, 145 L/h, 150 L/h, etc. Other specific point values within this range can also be selected, which will not be reiterated here.

Preferably, an adsorption time for the macroporous resin is 20-60 minutes, for example, it can be 20 min, 25 min, 30 min, 35 min, 40 min, 45 min, 50 min, 55 min, 60 min, etc.; a flow rate is 60-100 L/h, for example, it can be 60 L/h, 65 L/h, 70 L/h, 75 L/h, 80 L/h, 85 L/h, 90 L/h, 95 L/h, 100 L/h, etc. Other specific point values within this range can also be selected, which will not be reiterated here.

Preferably, in S5, the second eluent undergoes concentration to remove ethanol before standing.

Preferably, in S5, a standing temperature is 2-6° C., for example, it can be 2° C., 2.5° C., 3° C., 3.5° C., 4° C., 4.5° C., 5° C., 5.5° C., 6° C., etc.; a duration is 18-48 hours, for example, it can be 18 h, 19 h, 20 h, 21 h, 22 h, 24 h, 25 h, 26 h, 28 h, 30 h, 32 h, 35 h, 38 h, 40 h, 45 h, 48 h, etc. Other specific point values within this range can also be selected, which will not be reiterated here.

Preferably, in S5, the flocculant comprises a combination of gelatin, chitosan, and guar gum.

In the invention, it has been unexpectedly found that when gelatin, chitosan, and guar gum are simultaneously used as the flocculant, non-active impurities in the extract can be removed, resulting in a higher purity of the Ginkgo biloba extract obtained after treatment, which exhibits better effects, particularly in terms of antioxidant and moisturizing properties.

The flocculant is pretreated as follows before use: chitosan and guar gum are first mixed and stirred in a small amount of acetic acid aqueous solution until a paste is formed, then mixed with gelatin, and water is added and heated to swell.

Preferably, a mass ratio of the gelatin, the chitosan, and the guar gum is (0.5-2):1:(1-3); wherein, the aforementioned “0.5-2” can be, for example, 0.5, 0.6, 0.8, 1, 1.2, 1.4, 1.5, 1.6, 1.8, 2, etc.;

the aforementioned “1-3” can be, for example, 1, 1.2, 1.5, 1.8, 2, 2.2, 2.5, 2.8, 3, etc. Other specific point values within this range can also be selected, which will not be reiterated here.

Preferably, in S5, an amount of the flocculant is 0.05‰-0.1‰ of a weight of the filtrate; for example, it can be 0.05‰, 0.055‰, 0.06‰, 0.065‰, 0.07‰, 0.075‰, 0.08‰, 0.085‰, 0.09‰, or 0.095‰. Other specific point values within this range can also be selected, which will not be reiterated here.

Preferably, in S5, a mixing temperature is 50-60° C., for example, it can be 50° C., 51° C., 52° C., 53° C., 54° C., 55° C., 56° C., 57° C., 58° C., 59° C., 60° C., etc.; a duration is 10-30 minutes, for example, it can be 10 min, 12 min, 14 min, 15 min, 16 min, 18 min, 20 min, 25 min, 28 min, 30 min, etc. Other specific point values within this range can also be selected, which will not be reiterated here.

Preferably, a centrifugation speed of the flocculant is 2000-4000 rpm, for example, it can be 2000 rpm, 2250 rpm, 2500 rpm, 2750 rpm, 3000 rpm, 3250 rpm, 3500 rpm, 3750 rpm, 4000 rpm, etc.; a centrifugation time is 15-40 min, for example, it can be 15 min, 18 min, 20 min, 22 min, 25 min, 28 min, 30 min, 35 min, 40 min, etc. Other specific point values within this range can also be selected, which will not be reiterated here.

In a second aspect, the invention provides the Ginkgo biloba extract; the Ginkgo biloba extract is prepared by the preparation method of the first aspect.

In a third aspect, the invention provides an application of the Ginkgo biloba extract of the second aspect in preparing moisturizing and/or antioxidant products

Compared with the prior art, the invention has the following advantageous effects:

the invention develops a novel preparation method for Ginkgo biloba extract; creatively, it employs a specific extraction and separation purification process on the Ginkgo biloba treatment solution after enzymatic pretreatment; it combines with subsequent flocculant treatment for impurity removal; furthermore, it utilizes the composite enzyme combination (comprising β-glucosidase, xylanase, hemicellulase, and pectinase) for enzymatic hydrolysis; the combined use of these four enzymes allows for synergistic interaction and mutual positive influence, significantly enhancing the extraction efficiency of the Ginkgo biloba raw material and thereby increasing the active ingredients in the final extract product. The high-purity, high-activity Ginkgo biloba extract product is prepared, and exhibits excellent water solubility, which facilitates its application in cosmetics to enhance product efficacy in moisturizing, anti-inflammatory, and antioxidant properties.

### SPECIFIC EMBODIMENT OF THE INVENTION

To further elaborate on the technical means and effects adopted by the invention, the following will provide a further explanation of the technical solution of the invention in conjunction with the preferred embodiments of the invention. However, the invention is not limited to the scope of these embodiments.

In the following embodiments, unless otherwise specified, all reagents and consumables were purchased from conventional reagent manufacturers in the art; unless otherwise specified, the experimental methods and techniques used are conventional methods and techniques in the art.

The product information of β-glucosidase, xylanase, hemicellulase, and pectinase involved in the following embodiment are G824159 (Macklin), X875254 (Macklin), H8112 (Solarbio), and S10008 (Yuanye); gelatin, chitosan, and guar gum are all purchased from Shanghai Aladdin Reagent.

### Embodiment 1

This embodiment provides a Ginkgo biloba extract, its preparation method comprising the following steps:
(1) rough extraction step: grinding and crushing the Ginkgo biloba, then mixing with pure water at a solid-to-liquid ratio of 1:12 g/mL, mixing with a composite enzyme (composed of β-glucosidase, xylanase, hemicellulase, and pectinase in a mass ratio of 3:2:5:1); performing enzymatic hydrolysis at 52° C. for 1 hour, with an amount of composite enzyme being 0.3‰ of the crushed Ginkgo biloba; after the reaction, obtaining enzymatic hydrolysate, adding a 60% ethanol aqueous solution the enzymatic hydrolysate for reflux extracting, with a material-to-solvent ratio of Ginkgo biloba raw material to ethanol aqueous solution 1:8 g/mL; reflux extracting three times, with extraction times of 2 hours, 2 hours, and 1.5 hours respectively; combining the reflux liquids, concentrating under reduced pressure at 70° C. to remove ethanol, mixing the concentrated liquid with ten times its volume of pure water, heating and mixing at 80° C. for 30 minutes, cooling at 4° C. and letting it stand for 24 hours, then filtering to collect the filtrate to obtain a crude extract;
(2) first separation and purification step: loading the crude extract onto a D101 macroporous resin column for enriching, eluting with pure water at a flow rate of 100 L/h, and then eluting with a 75% ethanol aqueous solution to obtain a first eluent;
(3) fine extraction step: concentrating the first eluent under reduced pressure at 70° C. to remove ethanol, then drying the concentrated liquid at reduced pressure at 60° C. to obtain the Ginkgo biloba crude extract, mixing the Ginkgo biloba crude extract with a 75% ethanol aqueous solution and performing immersion extraction at 80° C. for 30 minutes, with a material-to-solvent ratio of 1:8 g/mL, cooling at 4° C. for 24 hours, then filtering to collect the filtrate to obtain a fine extract;
(4) second separation and purification step: concentrating the refined extract under reduced pressure at 70° C. to remove ethanol, mixing the concentrated liquid with twenty-five times its volume of pure water, then cooling at 4° C. for 24 hours, then filtering, loading the filtrate onto a resin column consisting of a polyamide resin column (100 L/h) and a DM130 macroporous resin column (80 L/h) in series, adsorbing for 30 minutes, eluting with pure water, and then analyzing with a 75% ethanol aqueous solution to obtain a second eluent;
(5) flocculation step: concentrating the second eluent under reduced pressure at 70° C. to remove ethanol, cooling at 4° C. and letting it stand for 24 hours, then filtering to collect the filtrate, mixing the filtrate with a flocculant (prepared by mixing chitosan and guar gum with three times their weight of 1% acetic acid aqueous solution until a paste is formed, then mixing with gelatin and adding seventy times their mass of water, swelling at 50° C. for 18 hours; a mass ratio of gelatin, chitosan, and guar gum is 1:1:1) for flocculation treating, with an amount of the flocculant being 0.08‰ of the filtrate's weight, at a temperature of 55° C. for 15 minutes to obtain a flocculation treatment liquid;
(6) drying step: centrifuging the flocculation treatment liquid at 2500 rpm for 35 minutes, taking a supernatant and filtering using a filter plate with a pore size of 1 μm, collecting the filtrate and drying to obtain the Ginkgo biloba extract.

### Embodiment 2

This embodiment provides a Ginkgo biloba extract, its preparation method comprising the following steps:
(1) rough extraction step: grinding and crushing the Ginkgo biloba, then mixing with pure water at a solid-to-liquid ratio of 1:8 g/mL, mixing with a composite enzyme (composed of β-glucosidase, xylanase, hemicellulase, and pectinase in a mass ratio of 1:3:4:1.5); performing enzymatic hydrolysis at 45° C. for 1.6 hours, with an amount of composite enzyme being 0.15‰ of the crushed Ginkgo biloba; after the reaction, obtaining enzymatic hydrolysate, adding a 40% ethanol aqueous solution the enzymatic hydrolysate for reflux extracting, with a material-to-solvent ratio of Ginkgo biloba raw material to ethanol aqueous solution 1:5 g/mL; reflux extracting two times, with each extraction time of 3 hours; combining the reflux liquids, concentrating under reduced pressure at 70° C. to remove ethanol, mixing the concentrated liquid with five times its volume of pure water, heating and mixing at 60° C. for 50 minutes, cooling at 6° C. and letting it stand for 48 hours, then filtering to collect the filtrate to obtain a crude extract;
(2) first separation and purification step: loading the crude extract onto a HPD100 macroporous resin column for enriching, eluting with pure water at a flow rate of 170 L/h, and then eluting with a 50% ethanol aqueous solution to obtain a first eluent;
(3) fine extraction step: concentrating the first eluent under reduced pressure at 70° C. to remove ethanol, then drying the concentrated liquid at reduced pressure at 70° C. to obtain the Ginkgo biloba crude extract, mixing the Ginkgo biloba crude extract with a 50% ethanol aqueous solution and performing immersion extraction at 50° C. for 50 minutes, with a material-to-solvent ratio of 1:5 g/mL, cooling at 6° C. for 48 hours, then filtering to collect the filtrate to obtain a fine extract;
(4) second separation and purification step: concentrating the refined extract under reduced pressure at 70° C. to remove ethanol, mixing the concentrated liquid with ten times its volume of pure water, then cooling at 6° C. for 48 hours, then filtering, loading the filtrate onto a resin column consisting of a polyamide resin column (150 L/h) and a DM130 macroporous resin column (100 L/h) in series, adsorbing for 50 minutes, eluting with pure water, and then analyzing with a 50% ethanol aqueous solution to obtain a second eluent;
(5) flocculation step: concentrating the second eluent under reduced pressure at 70° C. to remove ethanol, cooling at 6° C. and letting it stand for 48 hours, then filtering to collect the filtrate, mixing the filtrate with a flocculant (prepared by mixing chitosan and guar gum with three times their weight of 1% acetic acid aqueous solution until a paste is formed, then mixing with gelatin and adding seventy times their mass of water, swelling at 50° C. for 18 hours; a mass ratio of gelatin, chitosan, and guar gum is 0.5:1:3) for flocculation treating, with an amount of the flocculant being 0.05‰ of the filtrate's weight, at a temperature of 50° C. for 30 minutes to obtain a flocculation treatment liquid;
(6) drying step: centrifuging the flocculation treatment liquid at 2000 rpm for 40 minutes, taking a supernatant and filtering using a filter plate with a pore size of 1 μm, collecting the filtrate and drying to obtain the Ginkgo biloba extract.

### Embodiment 3

This embodiment provides a Ginkgo biloba extract, its preparation method comprising the following steps:
(1) rough extraction step: grinding and crushing the Ginkgo biloba, then mixing with pure water at a solid-to-liquid ratio of 1:14 g/mL, mixing with a composite enzyme (composed of β-glucosidase, xylanase, hemicellulase, and pectinase in a mass ratio of 5:4:2:3); performing enzymatic hydrolysis at 58° C. for 0.6 hours, with an amount of composite enzyme being 0.45‰ of the crushed Ginkgo biloba; after the reaction, obtaining enzymatic hydrolysate, adding a 85% ethanol aqueous solution the enzymatic hydrolysate for reflux extracting, with a material-to-solvent ratio of Ginkgo biloba raw material to ethanol aqueous solution 1:10 g/mL; reflux extracting four times, with extraction times of 1 hour, 2 hours, 2 hours and 1 hour respectively; combining the reflux liquids, concentrating under reduced pressure at 70° C. to remove ethanol, mixing the concentrated liquid with eighteen times its volume of pure water, heating and mixing at 100° C. for 20 minutes, cooling at 2° C. and letting it stand for 48 hours, then filtering to collect the filtrate to obtain a crude extract;
(2) first separation and purification step: loading the crude extract onto a AB-8 macroporous resin column for enriching, eluting with pure water at a flow rate of 120 L/h, and then eluting with a 85% ethanol aqueous solution to obtain a first eluent;
(3) fine extraction step: concentrating the first eluent under reduced pressure at 70° C. to remove ethanol, then drying the concentrated liquid at reduced pressure at 80° C. to obtain the Ginkgo biloba crude extract, mixing the Ginkgo biloba crude extract with a 90% ethanol aqueous solution and performing immersion extraction at 90° C. for 25 minutes, with a material-to-solvent ratio of 1:10 g/mL, cooling at 2° C. for 30 hours, then filtering to collect the filtrate to obtain a fine extract;
(4) second separation and purification step: concentrating the refined extract under reduced pressure at 70° C. to remove ethanol, mixing the concentrated liquid with forty times its volume of pure water, then cooling at 2° C. for 18 hours, then filtering, loading the filtrate onto a resin column consisting of a polyamide resin column (80 L/h) and a DM130 macroporous resin column (60 L/h) in series, adsorbing for 20 minutes, eluting with pure water, and then analyzing with a 90% ethanol aqueous solution to obtain a second eluent;
(5) flocculation step: concentrating the second eluent under reduced pressure at 70° C. to remove ethanol, cooling at 2° C. and letting it stand for 18 hours, then filtering to collect the filtrate, mixing the filtrate with a flocculant (prepared by mixing chitosan and guar gum with three times their weight of 1% acetic acid aqueous solution until a paste is formed, then mixing with gelatin and adding seventy times their mass of water, swelling at 50° C. for 18 hours; a mass ratio of gelatin, chitosan, and guar gum is 2:1:2) for flocculation treating, with an amount of the flocculant being 0.1‰ of the filtrate's weight, at a temperature of 60° C. for 10 minutes to obtain a flocculation treatment liquid;
(6) drying step: centrifuging the flocculation treatment liquid at 3500 rpm for 20 minutes, taking a supernatant and filtering using a filter plate with a pore size of 1 μm, collecting the filtrate and drying to obtain the Ginkgo biloba extract.

### Embodiment 4

This embodiment provides a Ginkgo biloba extract, using the Ginkgo biloba powder from Embodiment 1. The preparation method differs from that of Embodiment 1 only in that the amount of composite enzyme added is 1‰. The remaining steps and process parameters refer to Embodiment 1.

### Embodiment 5

This embodiment provides a Ginkgo biloba extract, using the Ginkgo biloba powder from Embodiment 1. The preparation method differs from that of Embodiment 1 only in that the amount of composite enzyme added is 0.05‰. The remaining steps and process parameters refer to Embodiment 1.

### Embodiment 6

This embodiment provides a Ginkgo biloba extract, using the second eluent from Embodiment 1. The preparation method differs from that of Embodiment 1 only in that the flocculant does not contain gelatin, and the reduced portions are proportionally distributed to chitosan and guar gum components. The remaining steps and process parameters refer to Embodiment 1.

### Embodiment 7

This embodiment provides a Ginkgo biloba extract, using the second eluent from Embodiment 1. The preparation method differs from that of Embodiment 1 only in that the flocculant does not contain chitosan, and the reduced portions are proportionally distributed to gelatin and guar gum components. The remaining steps and process parameters refer to Embodiment 1.

### Embodiment 8

This embodiment provides a Ginkgo biloba extract, using the second eluent from Embodiment 1. The preparation method differs from that of Embodiment 1 only in that the flocculant does not contain guar gum, and the reduced portions are proportionally distributed to chitosan and gelatin components. The remaining steps and process parameters refer to Embodiment 1.

### Comparative Example 1

This comparative example provides a Ginkgo biloba extract, using the Ginkgo biloba powder from Embodiment 1. The preparation method differs from that of Embodiment 1 only in that the composite enzyme does not contain β-glucosidase, and the reduced portions are proportionally distributed to xylanase, hemicellulase, and pectinase. The remaining steps and process parameters refer to Embodiment 1.

### Comparative Example 2

This comparative example provides a Ginkgo biloba extract, using the Ginkgo biloba powder from Embodiment 1. The preparation method differs from that of Embodiment 1 only in that the composite enzyme does not contain xylanase, and the reduced portions are proportionally distributed to β-glucosidase, hemicellulase, and pectinase. The remaining steps and process parameters refer to Embodiment 1.

### Comparative Example 3

This comparative example provides a Ginkgo biloba extract, using the Ginkgo biloba powder from Embodiment 1. The preparation method differs from that of Embodiment 1 only in that the composite enzyme does not contain hemicellulase, and the reduced portions are proportionally distributed to β-glucosidase, xylanase, and pectinase. The remaining steps and process parameters refer to Embodiment 1.

### Comparative Example 4

This comparative example provides a Ginkgo biloba extract, using the Ginkgo biloba powder from Embodiment 1. The preparation method differs from that of Embodiment 1 only in that the composite enzyme does not contain pectinase, and the reduced portions are proportionally distributed to β-glucosidase, xylanase, and hemicellulase. The remaining steps and process parameters refer to Embodiment 1.

### Comparative Example 5

This comparative example provides a Ginkgo biloba extract, using the Ginkgo biloba powder from Embodiment 1. The preparation method differs from that of Embodiment 1 only in that no enzymatic treatment is performed. The Ginkgo biloba powder is mixed with pure water and directly mixed with an ethanol aqueous solution for reflux extraction. The subsequent steps and process parameters refer to Embodiment 1.

### Comparative Example 6

This comparative example provides a Ginkgo biloba extract, using the first eluent from Embodiment 1. The preparation method differs from that of Embodiment 1 only in that no fine extraction and second separation and purification treatment are performed. The first eluent is used directly for subsequent flocculation and drying steps, with relevant steps and process parameters referring to Embodiment 1.

### Comparative Example 7

This comparative example provides a Ginkgo biloba extract, using the second eluent from Embodiment 1. The preparation method differs from that of Embodiment 1 only in that no flocculation treatment is performed. The second eluent is concentrated under reduced pressure at 70° C. to remove ethanol, cooled at 4° C. for 24 hours, filtered, and dried to obtain the Ginkgo biloba extract.

### Test Example 1

### Antioxidant Effect Test

### (1) DPPH Radical Scavenging

A 96-well plate was used, with three replicates for each group, and the system volume was 200 μL. Sample group: each extract sample of 100 μg from Embodiments 1-8 and Comparative Examples 1-7 was mixed with 100 μL of pure water, and then 100 μL of DPPH solution (0.1 mM) was added to the reaction system; control group: 100 μL of pure water was taken, followed by the addition of 100 μL of DPPH solution (0.1 mM). After constructing the reaction system, it was shaken in the dark for 10 minutes, and the absorbance at 517 nm was measured using a microplate reader.

Calculation method for scavenging rate: scavenging rate (%)=[(A0−Ax)/A0]×100%, where A0 is an absorbance of the control group and Ax is an absorbance of the sample group.

### (2) ABTS Radical Scavenging

Preparation of ABTS working solution: mixing ABTS with potassium persulfate solution to prepare a 7 mM ABTS stock solution, store in the dark for 12 hours, then mixing with 10 mM PBS (pH=7.4) to achieve an absorbance of 0.8 at 734 nm, resulting in the ABTS working solution.

A 96-well plate was used, with three replicates for each group, and the system volume was 200 μL. Sample group: each extract sample of 100 μg from Embodiments 1-8 and Comparative Examples 1-7 was mixed with 10 μL of pure water, and then 190 μL of ABTS working solution was added to the reaction system; control group: 10 μL of pure water was taken, followed by the addition of 190 μL of ABTS working solution. After constructing the reaction system, it was thoroughly shaken and incubated for 6 minutes, and the absorbance at 734 nm was measured using a microplate reader.

Calculation method for scavenging rate: scavenging rate (%)=[(A0−Ax)/A0]× 100%, where A0 is an absorbance of the control group and Ax is an absorbance of the sample group. The test results are shown in Table 1.

TABLE 1

| | DPPH - radical | ABTS - radical |
|---|---|---|
| Group | scavenging rate (%) | scavenging rate (%) |
| Embodiment 1 | 98.42 | 95.84 |
| Embodiment 2 | 96.88 | 93.69 |
| Embodiment 3 | 97.13 | 94.76 |
| Embodiment 4 | 93.06 | 90.75 |
| Embodiment 5 | 92.51 | 88.79 |
| Embodiment 6 | 91.85 | 89.64 |
| Embodiment 7 | 92.07 | 90.53 |
| Embodiment 8 | 89.76 | 88.34 |
| Comparative Example 1 | 85.64 | 83.59 |
| Comparative Example 2 | 86.66 | 84.37 |
| Comparative Example 3 | 85.98 | 83.91 |
| Comparative Example 4 | 86.04 | 84.00 |
| Comparative Example 5 | 81.59 | 80.25 |
| Comparative Example 6 | 74.32 | 71.38 |
| Comparative Example 7 | 79.63 | 76.55 |

Based on the data in the table,
(1) From Embodiments 1 to 3, it can be seen that the Ginkgo biloba extract prepared by the invention exhibits excellent DPPH and ABTS radical scavenging effects, demonstrating significant antioxidant activity, with the best effect observed in Embodiment 1.
(2) A comparison between Embodiment 1 and Embodiments 4-5 shows that when the amount of composite enzyme added is within a specific range, the antioxidant effect of the Ginkgo biloba extract produced is superior.
(3) A comparison between Embodiment 1 and Embodiments 6-8 indicates that using a flocculant composed of gelatin, chitosan, and guar gum to treat the extract can further remove inactive substances and other impurities from the extract, resulting in a high-purity active Ginkgo biloba extract.
(4) A comparison between Embodiment 1 and Comparative Examples 1~4 reveals that the combination of β-glucosidase, xylanase, cellulase, and pectinase used in the invention provides excellent enzymatic extraction effects for Ginkgo biloba. When any one of these four enzymes is absent in Comparative Examples 1-4, the antioxidant effect of the resulting Ginkgo biloba extract is lower than that of Embodiment 1, indicating that β-glucosidase, xylanase, cellulase, and pectinase have a synergistic promoting effect when extracting active components from Ginkgo biloba.
(5) A comparison between Embodiment 1 and Comparative Examples 5-7 shows that the specific extraction and purification method utilized in the present invention, which involves “enzymatic pretreatment-rough extraction-fine extraction-flocculation and impurity removal,” results in a high-purity and high-antioxidant activity Ginkgo biloba extract product.

### Test Example 2

### Anti-Inflammatory Effect Test

Testing Method: select logarithmic growth phase, well-formed RAW264.7 mouse macrophages are inoculated in DMEM culture medium containing 10% FBS (Foetal Bovine Serum) and incubated for 24 hours. Cells are seeded into a 24-well plate at a density of 2×10⁵ cells/well and further incubated for 2 hours in a 37° C., 5% CO₂ incubator. Cells are stimulated with 1 μg/mL lipopolysaccharide (LPS) for 24 hours to establish a cellular inflammation model.

0.5% of Ginkgo biloba extracts prepared from Embodiments 1-8 and Comparative Examples 1-7 are added to each group of cells. After incubating at 37° C. for 24 hours, the cell supernatant is collected, and the content of TNF-α, NO, and IL-6 in the collected supernatant is detected according to the steps outlined in the ELISA kit and nitric oxide detection kit instructions. The experimental results are analyzed using data processing software, and the results are shown in Table 2.

TABLE 2

| | TNF-α | NO | IL-6 |
|---|---|---|---|
| | inhibition | inhibition | inhibition |
| | rate (%) | rate (%) | rate (%) |
| Embodiment 1 | 60.7 | 68.4 | 63.8 |
| Embodiment 2 | 58.6 | 66.7 | 62.1 |
| Embodiment 3 | 59.0 | 67.1 | 62.9 |
| Embodiment 4 | 54.5 | 64.0 | 59.7 |
| Embodiment 5 | 53.8 | 62.7 | 57.4 |
| Embodiment 6 | 51.6 | 58.1 | 53.6 |
| Embodiment 7 | 50.8 | 57.9 | 51.8 |
| Embodiment 8 | 52.0 | 58.6 | 53.7 |
| Comparative Example 1 | 49.5 | 50.7 | 49.0 |
| Comparative Example 2 | 48.1 | 50.4 | 48.3 |
| Comparative Example 3 | 48.9 | 50.9 | 48.8 |
| Comparative Example 4 | 45.3 | 47.2 | 45.9 |
| Comparative Example 5 | 40.7 | 42.5 | 37.7 |
| Comparative Example 6 | 35.3 | 36.4 | 32.4 |
| Comparative Example 7 | 38.9 | 41.7 | 36.9 |

Based on the data in the table, it can be seen that the Ginkgo biloba extract prepared by the method provided in this invention exhibits excellent anti-inflammatory effects, significantly inhibiting the secretion of related pro-inflammatory factors. Among them, the anti-inflammatory effect of Embodiment 1 is superior to that of Embodiments 4-8 and Comparative Examples 1-7, indicating that the specific preparation conditions used for the Ginkgo biloba extract in this invention have varying degrees of influence on the anti-inflammatory efficacy of the final extract product.

### Test Example 3

### Moisturizing Effect Test

A total of 120 subjects aged between 28 and 40 years, with an average age of 34 years, were randomly divided into 15 groups. The subjects uniformly cleaned the inner sides of their left and right forearms and gently dried them with tissue. For each group, 6 areas measuring 3 cm×3 cm were marked on each person's forearm as test areas, with at least 2 cm spacing between each area. The results were averaged for statistical analysis. The test areas were applied with an appropriate amount of equally diluted samples from Embodiments 1-8 and Comparative Examples 1-7 (the Ginkgo biloba extract was mixed with pure water, resulting in a final extract concentration of 0.5% after dilution). Data collection from the subjects was arranged before application and 2 hours after application. Skin parameters were collected each time using a skin testing instrument (Corneometer CM825) to measure the increase rate of stratum corneum moisture content (%), where the increase rate=(moisture content after 2 hours of use-moisture content before use)/moisture content before use. The results are shown in Table 3.

TABLE 3

| Group | Moisture content increase rate (%) |
|---|---|
| Embodiment 1 | 58.1 |
| Embodiment 2 | 56.6 |
| Embodiment 3 | 57.0 |
| Embodiment 4 | 54.9 |
| Embodiment 5 | 52.1 |
| Embodiment 6 | 50.8 |
| Embodiment 7 | 51.9 |
| Embodiment 8 | 51.5 |
| Comparative Example 1 | 48.2 |
| Comparative Example 2 | 49.3 |
| Comparative Example 3 | 48.1 |
| Comparative Example 4 | 47.8 |
| Comparative Example 5 | 42.6 |
| Comparative Example 6 | 38.3 |
| Comparative Example 7 | 41.7 |

Based on the data in the table, it can be seen that the Ginkgo biloba extract prepared by the method provided in this invention exhibits excellent moisturizing effects, effectively maintaining the moisture content of the stratum corneum. The moisturizing effect of Embodiment 1 is superior to that of Embodiments 4-8 and Comparative Examples 1-7, indicating that the specific preparation conditions used for the Ginkgo biloba extract in this invention have varying degrees of influence on the moisturizing efficacy of the final extract product.

### Test Example 4

### Product Safety Testing

According to the inclusion criteria, 40 females and 40 males were selected to participate in the trial and randomly divided into 8 groups (5 males and 5 females in each group), each receiving a trial. An appropriate amount of extract samples prepared from Embodiments 1-8 (the extract was mixed with pure water to adjust the extract concentration to 4% before testing) was placed in the chambers of patch test tapes. The patch applicator containing the sample was adhered to the inner forearm skin of the subjects using medical tape and left on for 24 hours. After removing the patch applicator, skin reactions were observed 30 minutes after the indentations disappeared. For those with negative results, observations were repeated at 24 hours and 48 hours post-experiment to record skin reaction results. The degree of reaction was graded according to the standard shown in the following table.

| Degree of | Rating | |
|---|---|---|
| Reaction | Level | Skin Reaction |
| − | 0 | negative reaction |
| ± | 1 | suspicious reaction, with only slight |
| | | erythema |
| + | 2 | weak positive reaction (erythema reaction); |
| | | erythema, infiltration, edema, possible |
| | | papules |
| ++ | 3 | weak positive reaction (herpes reaction); |
| | | erythema, infiltration, edema, papules; the |
| | | reaction may extend beyond the test area |
| +++ | 4 | extremely strong positive reaction (confluent |
| | | herpes reaction); obvious erythema, severe |
| | | infiltration, edema, confluent herpes; the |
| | | reaction extends beyond the test area |

The results showed that the products prepared from Embodiments 1-8 exhibited negative reactions at both 24 hours and 48 hours post-testing, with no signs of irritation or erythema. It can be seen that the Ginkgo extract product obtained from this invention are non-irritating to the skin, showing no positive reactions, which demonstrates that the product has a high safety profile, low irritancy, and is relatively mild.

The applicant declares that the technical solution of this invention is illustrated through the above embodiments, but this invention is not limited to the above embodiments, that is, it does not mean that this invention must rely on the above embodiments to be implemented. Those skilled in the art should understand that any improvements to this invention, equivalent substitutions of raw materials for the product of this invention, additions of auxiliary components, and selection of specific methods, etc., all fall within the protection scope and disclosure scope of this invention.

The preferred embodiments of the invention have been described in detail above. However, the invention is not limited to the specific details of the above embodiments. Within the scope of the technical concept of the invention, various simple modifications can be made to the technical solution of the invention, and these simple modifications all fall within the protection scope of the invention.

It should also be noted that the various specific technical features described in the above embodiments can be combined in any suitable manner without contradiction. To avoid unnecessary repetition, the invention will not describe the various possible combinations separately.

1. A preparation method for Ginkgo biloba extract, comprising the following steps:
S1, rough extraction step: mixing Ginkgo biloba raw material with a composite enzyme for enzymatic hydrolysis; after the enzymatic hydrolysis is completed, using an ethanol aqueous solution for reflux extraction; concentrating the reflux liquid to remove ethanol; then mixing the concentrated solution with water; heating it; letting it stand; filtering the mixture; and
collecting filtrate to obtain a rough extract;
S2, first separation and purification step: enriching the rough extract using macroporous resin to obtain a first eluent;
S3, fine extraction step: concentrating and drying the first eluent to obtain Ginkgo biloba rough extract, then using an ethanol aqueous solution for hot immersion extraction, cooling, filtering, and collecting filtrate to obtain a fine extract;
S4, second separation and purification step: enriching the fine extract sequentially using polyamide resin and macroporous resin to obtain a second eluent;
S5, flocculation step: standing and filtering the second eluent, then collecting filtrate, mixing the filtrate with a flocculant to obtain a flocculation treatment liquid;
S6, drying step: centrifuging and filtering the flocculation treatment liquid, collecting and drying filtrate to obtain Ginkgo biloba extract;
wherein the composite enzyme comprises a combination of β-glucosidase, xylanase, hemicellulase, and pectinase.

2. The preparation method for Ginkgo biloba extract of claim 1, wherein a mass ratio of β-glucosidase, xylanase, hemicellulase, and pectinase is (1-5):(2-4):(2-6):(0.5-3).

3. The preparation method for Ginkgo biloba extract of claim 1, wherein before enzymatic hydrolysis, the Ginkgo biloba raw material is mixed with water at a solid-to-liquid ratio of 1:(8-15) g/mL;
preferably, an amount of the composite enzyme added is 0.1‰-0.5‰ of a mass of the Ginkgo biloba material;
preferably, a temperature for enzymatic hydrolysis is 40-60° C., and a duration is 0.5-2 hours.

4. The preparation method for Ginkgo biloba extract of claim 1, wherein in S1, a ratio of the Ginkgo biloba raw material to the ethanol aqueous solution is 1:(4-10) g/mL;
preferably, in S1, a volume percentage of ethanol in the ethanol aqueous solution is 30%-90%;
preferably, in S1, the number of reflux extraction cycles is 2-5 times, with each reflux extraction lasting 1-3 hours;
preferably, in S1, a volume ratio of the concentrated solution to water is 1:(2-20);
preferably, in S1, a temperature for mixing with water and heating is 50-100° C., for a duration of 20-60 minutes;
preferably, in S1, a temperature for standing is 2-6° C., for a duration of 18-48 hours.

5. The preparation method for Ginkgo biloba extract of claim 1, wherein in S2, a type of the macroporous resin comprises any one of AB-8 type, D101 type, or HPD100 type;
preferably, in S2, the enrichment specifically comprises: loading the rough extract onto the macroporous resin for adsorption, eluting with water, analyzing with a 40%-90% ethanol aqueous solution, and collecting the eluent;
preferably, an elution flow rate is 120-180 L/h.

6. The preparation method for Ginkgo biloba extract of claim 1, wherein in S3, a temperature for concentration and drying is 60-80° C.;
preferably, in S3, a ratio of the Ginkgo biloba rough extract to the ethanol aqueous solution is 1:(4-10) g/mL;
preferably, in S3, a volume percentage of ethanol in the ethanol aqueous solution is 40%-90%;
preferably, in S3, a temperature for the hot immersion extraction is 40-100° C., for a duration of 20-60 minutes;
preferably, in S3, a cooling temperature is 2-6° C., for a duration of 18-48 hours.

7. The preparation method for Ginkgo biloba extract of claim 1, wherein in S4, the type of macroporous resin comprises DM130;
preferably, the enrichment specifically comprises: sequentially loading the fine extract onto the polyamide resin and macroporous resin for adsorption, eluting with water to remove impurities, analyzing with a 40%-90% ethanol aqueous solution, and collecting the eluent;
preferably, before loading, the fine extract undergoes concentration to remove ethanol, mixing the concentrated solution with water, cooling, and filtering to collect the filtrate;
preferably, a volume ratio of the concentrated solution to water is 1:(10-40);
preferably, a cooling temperature is 2-6° C., for a duration of 18-48 hours; preferably, an elution flow rate for the polyamide resin is 80-150 L/h;
preferably, an adsorption time for the macroporous resin is 20-60 minutes, with a flow rate of 60-100 L/h.

8. The preparation method for Ginkgo biloba extract of claim 1, wherein in S5, the second eluent undergoes concentration to remove ethanol before standing;
preferably, in S5, a standing temperature is 2-6° C., for a duration of 18-48 hours;
preferably, in S5, the flocculant comprises a combination of gelatin, chitosan, and guar gum;
preferably, a mass ratio of the gelatin, the chitosan, and the guar gum is (0.5-2):1:(1-3);
preferably, in S5, an amount of the flocculant is 0.05‰-0.1‰ of a weight of the filtrate;
preferably, in S5, a mixing temperature is 50-60° C., for a duration of 10-30 minutes.

9. A Ginkgo biloba extract, prepared by the preparation method of claim 1.

10. An application of the Ginkgo biloba extract of claim 9 in preparing moisturizing and/or antioxidant products.
